(19) United States
(12) Patent Application Publication
(10) Pub. No.: US 2018/0164108 A1
(43) Pub. Date: Jun. 14, 2018

(54) STRESS BASED NAVIGATION ROUTING

(71) Applicant: Intel Corporation, Santa Clara, CA (US)

(72) Inventors: Tawfik Rahal-Arabi, Tigard, OR (US); Mark MacDonald, Beaverton, OR (US)

(21) Appl. No.: 15/374,878

(22) Filed: Dec. 9, 2016

(51) Int. Cl. G01C 21/34; A61B 5/16; A61B 5/18; A61B 5/00

(52) U.S. Cl. CPC G01C 21/3484; G01C 21/3461; A61B 5/6893; A61B 5/165; A61B 5/18; G01C 21/3492

(57) ABSTRACT

In one example, a navigation routing system includes at least one processor to provide a navigation route based on one or more stress levels of a driver of a vehicle.

### TECHNICAL FIELD

This disclosure relates generally to safety and/or stress based navigation routing.

### BACKGROUND

Navigation systems such as GPS (Global Position Satellite) systems can offer various tools for mapping and route planning. For example, navigation systems might provide routes for situations such as the shortest route, least traffic, avoiding highways, etc.

### BRIEF DESCRIPTION OF THE DRAWINGS

The following detailed description may be better understood by referencing the accompanying drawings, which contain specific examples of numerous features of the disclosed subject matter.

FIG. 1 illustrates a safety based and/or stress based navigation routing system;

FIG. 2 illustrates a safety based and/or stress based navigation routing system;

FIG. 3 illustrates driver stress profile optimization;

FIG. 4 illustrates driver stress profile updating;

FIG. 5 illustrates a block diagram of a processor and one or more non-transitory computer readable media for safety and/or stress based navigation;

FIG. 6 illustrates a block diagram of a computing device.

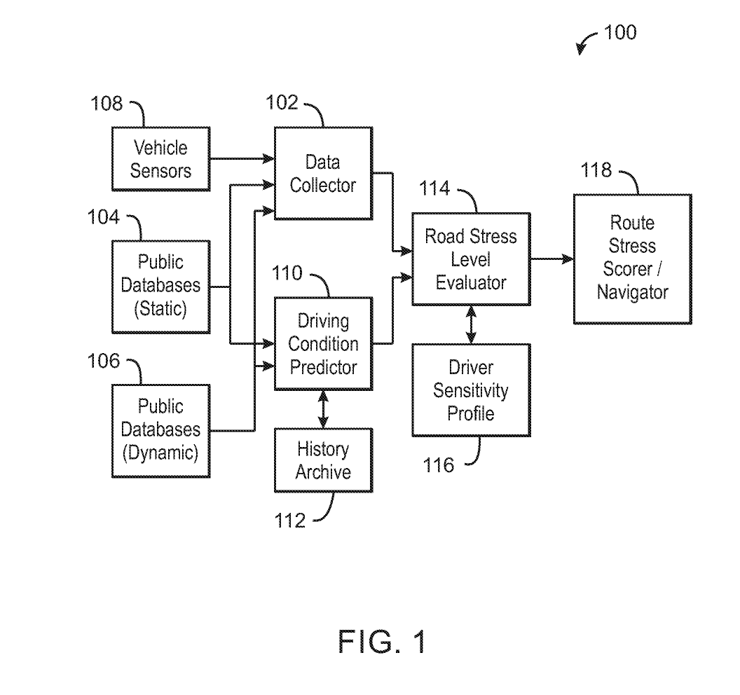

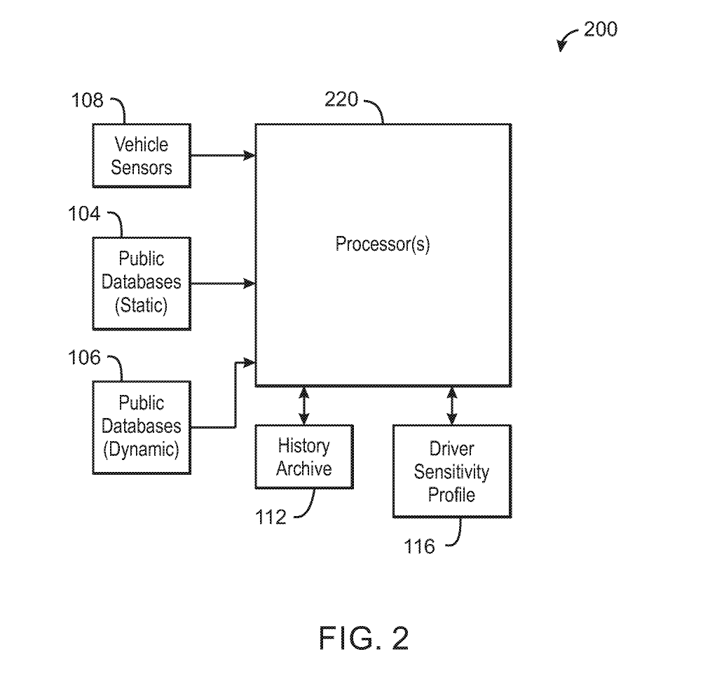

In some cases, the same numbers are used throughout the disclosure and the figures to reference like components and features. In some cases, numbers in the 100 series refer to features originally found in FIG. 1; numbers in the 200 series refer to features originally found in FIG. 2; and so on.

### DESCRIPTION OF THE EMBODIMENTS

Some embodiments relate to stress based navigation routing. Some embodiments relate to safety based navigation routing. Some embodiments relate to stress based navigation routing, leading to higher safety conditions for the driver and/or passengers of a vehicle. That is, some embodiments relate to safety and/or stress based navigation routing. Some embodiments relate to Global Positioning System (GPS) routing.

As discussed above, navigation systems such as GPS (Global Position Satellite) systems can offer various tools for mapping and route planning. For example, navigation systems might provide routes for situations such as the shortest route, least traffic, avoiding highways, etc. However, many drivers might prefer obtaining route information based on a higher safety and/or lower stress experience. For example, senior citizens, retirees, or new drivers may be intimidated by certain driving conditions or routes where driving behavior is generally more aggressive, and would prefer to take a slower and/or longer route that provides more safety and/or less driving stress.

Many drivers might prefer obtaining route information based on a safer and/or lower stress experience. For example, senior citizens, retirees, or new drivers may be intimidated by certain driving conditions or routes where driving behavior is generally more aggressive, and would prefer to take a slower and/or longer route that provides less driving stress. For example, according to some embodiments, a navigation system provides safety and/or stress based navigation so that a driver can avoid difficult merges, narrow streets, high crime neighborhoods, and/or other areas for a safer and/or less stressful driving experience.

In some embodiments, a GPS, smart car, and/or other route planning device is used to provide safety and/or stress based navigation routing. In some embodiments, public databases can be accessed and data from the public databases can be used, for example, in addition to internal and/or external vehicle sensor measurements to generate a route plan for a driver that optimizes a high safety level and/or minimum stress level for that driver.

Some embodiments relate to a navigation system such as a navigation system on a computer, a phone, a car, etc. In some embodiments, in addition to available options such as suggesting the shortest route or the fastest route, the navigation system identifies to the user a safe driving route and/or a low stress driving route that is optimized for safety and/or low driver stress level. The safe driving route and/or low stress driving route can be implemented in response to many different data points, for example, including static public databases, dynamic public databases, internal and external sensor measurements, driving history data, driver stress sensitivity information, etc. Many factors that can cause less safe driving and/or driving stress that the navigation system (and/or smart vehicle) can consider in determining a safe and/or low stress driving route according to some embodiments. For example, traffic patterns can be considered in some embodiments. These traffic patterns can include, for example, current accident or current heavy traffic locations as well as historic data such as which locations, intersections, etc. have high traffic accident rates. These types of information can be accessed through various accident databases (for example, databases that are compiled and available from state, counties, police stations, etc). In some embodiments, the navigation system can be aware of unsafe, high risk and/or high stress travel areas in order to provide travel routes that are safer, lower stress and/or lower risk routes. Additionally, the safe and/or low stress route can be optimized around (or tailored to) avoiding situations and/or locations that are particularly dangerous and/or stressful to the individual driver (and/or passengers) of the vehicle.

In some embodiments, calendar and/or historical information may be used in determining a low stress route. For example, for an elderly person that wants to avoid stress due to locations, traffic patterns such as rush hour, etc. the person may historically have gone to pick up grandchildren from school leaving and arriving hours earlier than necessary in order to avoid stress. However, in some embodiments, in addition to current accident and traffic data, the safe and/or low stress routing can use historical and calendar information, for example, to identify an equally low stress routing that would not require the person to drive and sit for a few hours in the school parking lot waiting for grandchildren to finish school. In some embodiments in which calendar integration is used, even if a grandparent does not like rush hour driving, instead of heading to a location to pick grandchildren up several hours, early, a safe route and/or low stress route is provided that does not require such a long wait in order to avoid traffic.

In some embodiments, higher safety and/or low stress routing can be individualized using current individual safety and/or stress information about the driver (and/or passengers) using sensors to detect information such as heart rate, blood pressure, etc., but additionally can be individualized to past experiences of the particular driver (and/or passenger). For example, if the last time the driver (and/or passenger) went through a particular intersection or on a particular portion of a road, etc. the sensors indicated an increased stress and/or less safe driving, those areas could be avoided in the future (or could be avoided at certain times if there were only certain times or days that the individual was less safe and/or more stressed at certain routing points). In some embodiments, a rain sensor can be used (for example, outside the vehicle) to determine if rain conditions exist. In some embodiments, sensors of the vehicle can be used to determine if the vehicle operated in a way that might indicate a lower vehicle safety level and/or a higher driver stress level under certain conditions (for example, while the rain conditions existed, or while slippery road conditions existed based on input from a weather web site or database, etc.).

Many different aspects of driver condition prediction and user profile information can be used to determine safety, stress and/or anticipated stress levels and redirect the route accordingly. In some embodiments, a driver sensitivity profile can be used, which stores information about the driver's (and in some embodiments passengers) sensitivity to certain types of driving situations. For example, if previous sensing (video or other types of sensing) indicate that the driver was less safe and/or more stressed in certain types of situations, that information can be considered. For example, if the driver sensitivity profile shows that the driver was less safe and/or more stressed when driving with children present in the back seat (and/or an identification is made that the driver is about to pick up children from school) then the low stress routing can route the driver in a less stressful route (for example, using back roads or less stressful roads with less driving stress involved after the driver is scheduled to pick children up and drive to the next location). Therefore, in some embodiments, calendar integration with monitoring of driving situations over time can be used to provide safer and/or less stressful driving routes based on many different safety and/or stress factors, past history, etc.

In some embodiments, high safety and/or low stress navigation routing determines information about (and/or learns about) particular drivers over time (and in some cases, particular drivers and different stress levels based on different passengers and/or types of passengers in the vehicle) and then provides safer routes and/or lower stress routes in situations where the driver is likely to become more stressed. The routing determines (and/or learns) characteristics of the driver that help to provide safer and/or low stress route suggestions for that particular driver. For example, if certain situations cause a driver to make sudden lane changes, sudden starts or stops, or otherwise make a driver appear to be tense, those aspects can be factored into making safer and/or low stress navigation routing suggestions.

In some embodiments, additional sensor devices can be integrated and provide input to the high safety and/or low stress navigation routing system. For example, health devices such as shoes, hats, watches, or other wearable devices that help to measure driver stress factors such as blood pressure and/or pulse rate can provide data into the system to help make stress routing decisions. These factors (either historical or current measurements) can be used to help route the driver in a high safety and/or low stress way in order to reduce the impact of stress during the drive. Other current and/or historical stress measurement factors that can be used can include how hard the person is gripping the steering wheel, whether the person's hands are at a particular arrangement on the steering wheel (such as “ten and two” or steering in a more relaxed way with only one hand or even using other body parts such as the knees to steer), other types of factors, etc. Sensors to monitor this type of activity could include pressure sensors, camera sensors, sound sensors, etc.

In some embodiments, navigation routing is implemented based on one or more stress levels (for example, based on one or more stress levels of a driver of a vehicle and/or based on one or more stress levels of one or more passengers of the vehicle). In some embodiments, the one or more stress levels are calculated, stored, and/or measured. In some embodiments, the one or more stress levels are based on past experiences, current situations, user input, database information, calendar information, prediction, and/or other calculated, stored and/or measured ways.

FIG. 1 illustrates a safety based and/or stress based navigation routing system 100. In some embodiments, safety and/or stress based navigation routing system 100 is related to and/or implemented partially or completely in one or more of a GPS navigation system, a vehicle, a smart vehicle, a smart car, a phone, a tablet, a mobile device, a laptop computer, and/or a desktop computer, etc. System 100 includes a data collector 102, one or more static public database(s) 104, one or more dynamic public database(s) 106, one or more vehicle sensors 108, a driving condition predictor 110, a history archive 112, a road stress level evaluator 114 (in some embodiments, road stress level evaluator 114 is a road safety level evaluator and/or a road stress level evaluator), a driver sensitivity profile 116, and a route stress scorer/navigator 118 (in some embodiments, route stress scorer/navigator 118 is a route safety scorer/navigator and/or a route stress scorer/navigator). In some embodiments, one or more of data collector 102, driving condition predictor 110, road stress level evaluator 114, and/or route stress scorer/navigator 118 can be implemented using one or more processors.

Data collector 102 collects and aggregates data from multiple sources, including, for example, static public database(s) 104, dynamic public database(s) 106, and vehicle sensors 108. The collected data can include, for example, both potential stress factors (for example, environmental conditions that might lead to driver stress) and potential stress indicators (for example, direct or indirect measurements of a driver's current stress levels). In some embodiments, a GPS (Global Positioning System), smart car, and/or route planning tool is used to access public databases such as static public databases 104 and/or dynamic public databases 106 in order to generate a route plan for a driver that optimizes a driving route for a maximum safety level and/or a minimum stress level of the driver. This optimization can be implemented based on a number of different stress level factors that are taken into account.

Static public databases 104 can include, for example, one or more of crime rate, police, county record, and street infrastructure databases, etc. In some embodiments, the static public databases 104 can be updated periodically (for example, once a month), and can include information such as the number of accidents at particular intersections within the past year, month, week, etc. These databases are remotely accessed at a particular periodic rate (for example, once a month, once a week, etc.) or can be pushed to the navigation system at a non-periodic time in some circumstances, and are then accessed locally by the system 100.

In some embodiments, potential safety factors and/or potential stress factors that can be accessed from static public databases 104 can include neighborhood crime rates (for example, from police, sheriff, state, county, city, etc. databases), intersections, streets, and/or portions of streets with high accident occurrence rates (for example, from police databases), how many accidents have occurred at a particular locations or intersection over the past year, streets or certain portions of streets with high rates of speeding drivers (for example, from police or traffic flow databases), street widths (for example, from county records databases or from Google® street view), roundabout or other unusual street traffic patterns (for example, from map databases), blind turns or intersections (for example, from Google® street view and/or police accident databases), and/or locations with a high concentration of bicyclists (for example, from bike lane databases).

Similar to static public databases 104, dynamic public databases 106 relate to potential stress factors. Dynamic public databases 106 can be dynamic public information sources such as, for example, traffic alert databases, weather reports, other hazard information, event information, etc. The dynamic public databases 106 can provide, for example, data relating to construction, traffic congestion, accidents that have recently occurred, special events such as a parade or sporting event occurring in a particular area, etc. In some embodiments, dynamic public databases are connected on the fly (for example, via cloud connection).

In some embodiments, potential stress factors that can be accessed from dynamic public databases 106 can include construction and/or traffic pattern changes (for example, from mapping databases), current accident locations (for example, from traffic alert databases), weather conditions or events (for example, from weather databases such as NOAA or National Oceanic and Atmospheric Administration databases), scheduled events (for example, parade, concert or sporting events from various internet sources), and a drivers personal calendar data (although, since a drivers personal calendar data is not typically public data, this information can also come from a non-public source such as the person's private calendar resident in the cloud or on a device of the person).

Vehicle sensors 108 can include various sensors on, in or near the vehicle, the driver, and/or other vehicle occupants. These sources could include smartphone, camera, wearable, and/or other types of vehicle and/or personal sensors. Data provided by vehicle sensors 108 can include potential stress factors such as conditions internal and external to the vehicle as well as potential stress indicators such as direct measurements associated with the driver's current stress level.

Potential stress factors from dynamic sensor measurements from vehicle sensors 108 can include, for example, audio captures such as audio recordings from inside the vehicle. For example, if children are crying or bickering in the back seat, an argument is occurring inside the vehicle, or a stressful phone call is being placed inside the vehicle, etc. Other potential stress indicators from dynamic sensor measurements from vehicle sensors 108 can include, for example, vehicle camera data (for example, picking up driving hazards or conditions not reported in the above databases, such as the vehicle approaching a group of bicyclists).

Potential stress indicators from dynamic sensor measurements from vehicle sensors 108 can include, for example, driver pulse and blood pressure (measured by a wearable device, through the steering wheel, or a seat based biometric sensor, for example), driver hand position (for example, measured by steering wheel proximity sensors identifying things such as one handed driving which implies less stress than two handed driving, driver with hands at 10 and 2 positions implies more stress than one handed driving or two hands is more casual locations, etc.), time history and angle of the steering wheel, driver posture (for example, measured by seat pressure sensors and/or infrared or IR cabin sensor identifying conditions such as where nervous drivers leans forward and/or sits upright more than non-nervous drivers), fluidity of steering (for example, measured by a steering wheel position indicator identifying situations such as jerky steering movements that imply driver stress), cabin audio detection, cameras, and/or driver eyesight direction monitors identifying changes using gaze/eye tracking cameras in the cabin in order to obtain an indication of stress, etc. Additionally, in some embodiments, potential stress indicators can be measured using other current or future emerging stress measurement devices (for example, using EKG, etc.).

Driving condition predictor 110 can also poll similar potential stress factor data from static public databases 104 and dynamic public databases 106 as that used by data collector 102. In some embodiments, driving condition predictor 110 and history archive 112 work together to anticipate stress conditions. In some embodiments, driving condition predictor 110 examines various potential stress factors and makes predictions about the stress environment of the driver in the near future based on an archive of stress histories stored in history archive 112. In some embodiments, history archive 112 predicts an environmental condition at a particular time and/or place. For example, driving condition predictor 110 could anticipate that the driver is on their way to pick up a carpool load of noisy children that might cause stress in the later part of the journey. In some embodiments, history archive 112 can provide data used to predict environmental conditions such as, for example, anticipating a traffic jam near a school at the beginning or end of a school day, or noting that every time the driver goes to the school the cabin of the vehicle gets noisy, etc. In some embodiments, history archive 112 can be local (for example at the vehicle). In some embodiments history archive 112 can be implemented remotely (for example, in the cloud). In some embodiments, history archive 112 can connect with sensors (for example, with vehicle sensors 108), and can use machine learning algorithms, for example, to extract stress levels based on time histories, etc.

In some embodiments, road stress level evaluator 114 determines effective stressfulness of particular roads to the driver in response to data from data collector 102, data from driving condition predictor 110, and/or from driver sensitivity profile 116. In some embodiments, a road stress level evaluator 114 is implemented separately for each individual driver, and/or evaluates road stress specific to a particular driver. In some embodiments, road stress level evaluator 114 and driver sensitivity profile 116 can work together to fine tune a driver's stress profile. In some embodiments, driver sensitivity profile 116 can predict (and/or be used to predict) sensitivity of the specific driver to particular conditions. In some embodiments, driver sensitivity profile 116 can store various response sensitivities of the driver based on, for example, a history of measured driver stress indicators. In some embodiments, road stress evaluator 114 and driver sensitivity profile 116 can, for example, use information such as situations that might cause the driver to make a sudden lane change, etc., and how those situations might be avoided. In some embodiments, driver sensitivity profile 116 is implemented remotely in the cloud, and/or implemented in a manner that moves back and forth to the cloud.

In some embodiments, driver sensitivity profile 116 is continuously updated as more data is collected relating to the particular driver associated with the profile. In some embodiments, for example, driver sensitivity profile 116 can store features such as, noise in the car bothers that driver or doesn't bother that driver (and/or distract that driver). In some embodiments, processing of driver sensitivity profile 116 information can occur locally (for example, at the vehicle) and/or remotely (for example, in the cloud). In some embodiments, the identity of the particular driver is determined, for example, using one or more of facial recognition, a key fob, other security devices, etc. in order to uniquely identify the correct driver profile to be used based on the current driver. In some embodiments, driver sensitivity profile 116 can be local (for example, at the vehicle). In some embodiments driver sensitivity profile 116 can be implemented remotely (for example, in the cloud).

In some embodiments, current data output from vehicle sensors 108 is used by road stress level evaluator 114 to determine stress factors currently effecting the driver. In addition, in some embodiments, road stress level evaluation 114 considers how previous experiences might affect the driver over time. For example, driving condition predictor 110 and driver sensitivity profile 116 can provide information including things that a driver can choose (such as particular stressful types of driving conditions that the driver wants to avoid), but can also provide things determined and/or learned by monitoring the driver over time. For example, in some embodiments, calendar integration can be used with driver monitoring and learning about the particular driver over time. For example, personal responses of the driver can be learned over time. For example, in some embodiments, factors such as that the driver gets nervous when children are in the car, and the drivers calendar indicates that the driver is picking up children after school, system 100 can be used to provide a less stressful road choice after the driver picks up the children based on integration with the drivers' calendar.

Route stress scoring/navigation device 118 is used to optimize a desired safer and/or stress optimized route among various different road options and their associated stress scores to determine a recommended route. In some embodiments, driving route planning is implemented in a manner that can avoid and/or minimize a presence of key potential stress factors on a driving route in an optimized manner.

In some embodiments, data collector 102, driving condition predictor 110, road stress level evaluator 114, and/or route stress scoring/navigation device 118 may be implemented using one or more processor. In some embodiments, those devices (and/or one or more processor) are implemented at the vehicle location. In some embodiments, those devices (and/or one or more processor) are implemented at the cloud. In some embodiments, devices of FIG. 1 may be local (for example, at the vehicle). In some embodiments, devices of FIG. 1 may be remote (for example, at the cloud). In some embodiments, some or all processing implemented in system 100 can be implemented locally (for example, at the vehicle). In some embodiments, some or all processing implemented in system 100 can be implemented remotely (for example, in the cloud). For example, in some embodiments, monitoring can be implemented using an Intel® Curie solution, and various data streams can be sent remotely for processing (for example, sent to the cloud and processed at the cloud).

FIG. 2 illustrates a safety based and/or stress based navigation routing system 200. In some embodiments, safety and/or stress based navigation routing system 200 is related to and/or implemented partially or completely in one or more of a GPS navigation system, a vehicle, a smart vehicle, a smart car, a phone, a tablet, a mobile device, a laptop computer, and/or a desktop computer, etc. System 200 includes one or more static public database(s) 104, one or more dynamic public database(s) 106, one or more vehicle sensor(s) 108, a history archive 112, a driver sensitivity profile 116, and one or more processor(s) 220. In some embodiments, processor(s) 220 provides stress based routing. In some embodiments, processors(s) 220 provide some or all of the functionality similar to that performed by one, some, or all of data collector 102, driving condition predictor 110, road stress level evaluator 114, and/or route stress scorer/navigator 118. In some embodiments, history archive 112 and/or driver sensitivity profile 116 are included within processor(s) 220.

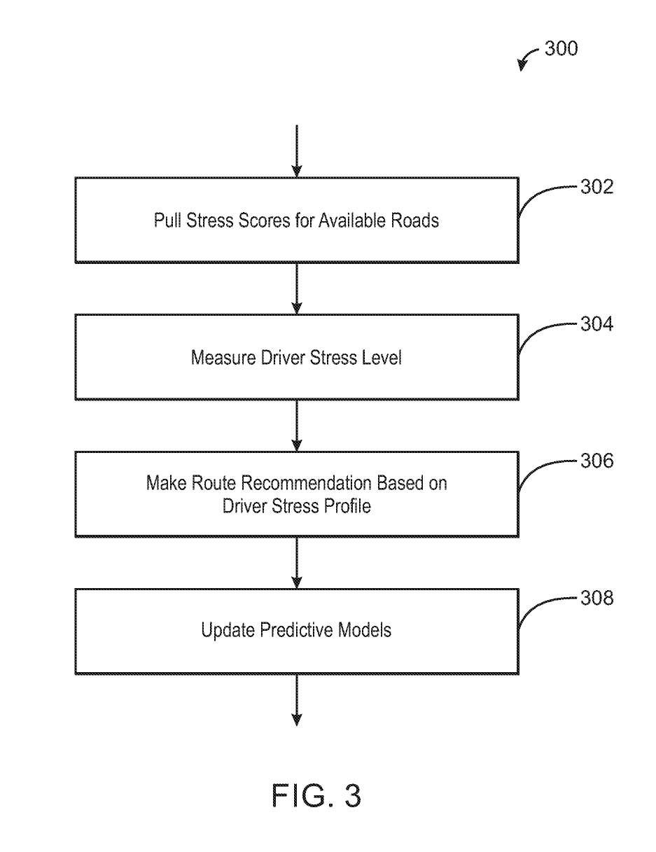

FIG. 3 illustrates driver profile optimization 300 according to some embodiments. In some embodiments, driver profile optimization 300 can be implemented using system 100 of FIG. 1 (and/or using a portion of system 100 of FIG. 1), and/or can be implemented using system 200 of FIG. 2 (and/or using a portion of system 200 of FIG. 2). In some embodiments, driver profile optimization 300 can be implemented using one or more processor (for example, using one or more processors 220). In some embodiments, driver profile optimization 300 can be implemented using software running on one or more processor (for example, using software running on one or more processors 220). In some embodiments, driver profile optimization 300 is implemented at the vehicle location. In some embodiments, driver profile optimization 300 can be implemented elsewhere (for example, it is implemented in the cloud). In some embodiments, driver profile optimization 300 can be implemented partially at the vehicle and partially elsewhere (for example, partially implemented at the vehicle and partially implemented in the cloud). In some embodiments, driver profile optimization 300 can be implemented using one or more of data collector 102, driving condition predictor 110, history archive 112, road stress level evaluator 114, drive sensitivity profile 116 and/or route stress scorer/navigator 118.

At block 302 safety and/or stress scores for available roads are obtained (for example, in some embodiments, they are pulled from a database such as one or more of databases 104, databases 106, history archive 112, and/or driver sensitivity profile 116 of FIG. 1). In some embodiments, at block 302, road data can be pulled from a database. One or more driver stress levels (and/or safety levels) are measured at 304 (for example, in some embodiments, they are measured using one or more of data collector 102, driving condition predictor 110, road stress level evaluator 114 and/or route stress scorer/navigator 118 of FIG. 1). Driver profile optimization 300 then makes a recommendation at 306 that is based on a safety and/or driver stress profile (for example, in some embodiments, using road stress level evaluator 114 based on data from driver sensitivity profile 116 of FIG. 1). Driver profile optimization 300 updates predictive models at 308 (for example, using driving condition predictor 110 and/or road stress level evaluator 114 of FIG. 1).

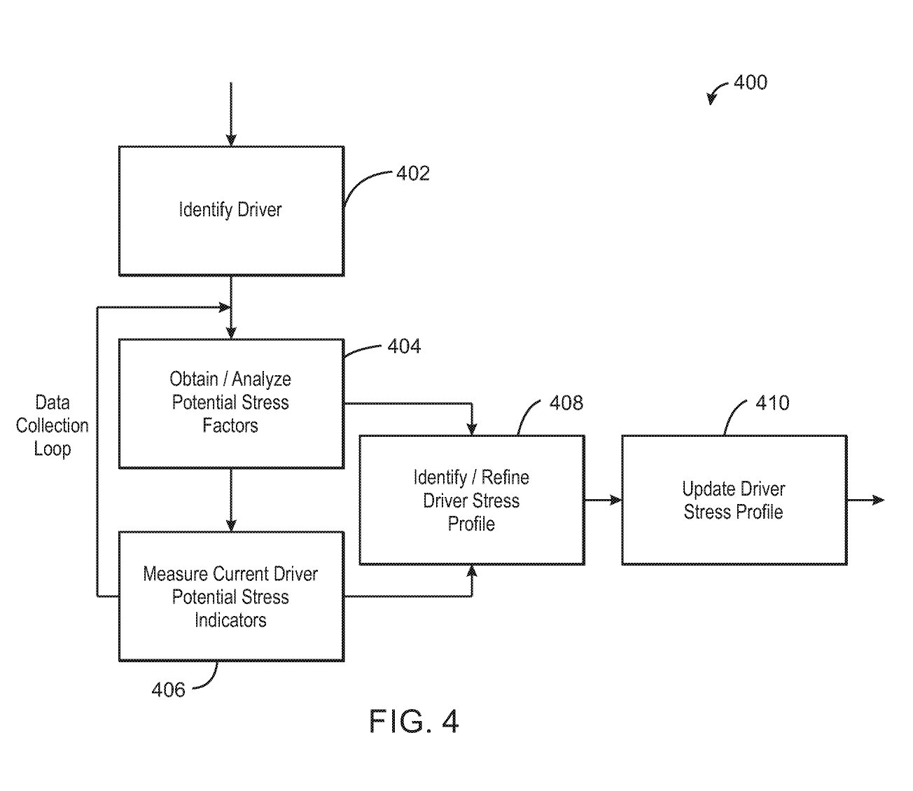

FIG. 4 illustrates safety profile updating and/or driver stress profile updating 400 according to some embodiments. In some embodiments, safety and/or driver stress profile updating 400 can be implemented using system 100 of FIG. 1 (and/or using a portion of system 100 of FIG. 1), and/or can be implemented using system 200 of FIG. 2 (and/or using a portion of system 200 of FIG. 2). In some embodiments, safety and/or driver stress profile updating 400 can be implemented using one or more processor (for example, using one or more processors 220). In some embodiments, safety and/or driver stress profile updating 400 can be implemented using software running on one or more processor (for example, using software running on one or more processor 220). In some embodiments, safety and/or driver stress profile updating 400 can be implemented at the vehicle location. In some embodiments, safety and/or driver stress profile updating 400 can be implemented elsewhere (for example, it is implemented in the cloud). In some embodiments, safety and/or driver stress profile updating 400 can be implemented partially at the vehicle and partially elsewhere (for example, partially implemented at the vehicle and partially implemented in the cloud). In some embodiments, safety and/or driver stress profile updating 400 can be implemented using one or more of data collector 102, driving condition predictor 110, history archive 112, road stress level evaluator 114, drive sensitivity profile 116 and/or route stress scorer/navigator 118.

At block 402 a driver of a vehicle is identified (for example, in some embodiments, using facial recognition, a key fob, and/or some other method such as a security metric). In some embodiments, driver identification at block 402 can be implemented using one or more of data collector 102, driving condition predictor 110, road stress level evaluator 114 and/or route stress scorer/navigator 118. In some embodiments, block 404 and block 406 are included in a data collection loop. Potential safety and/or stress factors are obtained and/or analyzed at block 404. For example, in some embodiments, block 404 downloads and/or analyzes current location potential safety and/or stress factors from available databases. For example, in some embodiments, block 404 uses one or more of data collector 102, driving condition predictor 110, road stress level evaluator 114 and/or route stress scorer/navigator 118 of FIG. 1 to analyze and/or obtain current location potential safety and/or stress factors. In some embodiments, block 404 obtains and/or downloads current location potential safety and/or stress factors from a database such as one or more of databases 104, databases 106, vehicle sensors 108, history archive 112, and/or driver sensitivity profile 116 of FIG. 1. In some embodiments, current driver potential safety and/or stress indicators are measured at block 406 (for example, using one or more of vehicle sensors 108, data collector 102, driving condition predictor 110, road stress level evaluator 114 and/or route stress scorer/navigator 118 of FIG. 1). A driver safety and/or stress profile is identified and/or refined at block 408 (for example, using machine learning, neural net, and/or other algorithms). In some embodiments, block 408 is performed using one or more elements of FIG. 1 such as road stress evaluator 114. At block 410 a safety and/or driver stress profile is updated (for example, it is periodically updated). For example, in some embodiments, block 410 updates a safety and/or driver stress profile in a navigation system such as a navigation system in a vehicle (for example, navigation system 100 of FIG. 1). In some embodiments, block 410 is implemented using a device such as one or more of data collector 102, driving condition predictor 110, road stress level evaluator 114 and/or route stress scorer/navigator 118 of FIG. 1.

In some embodiments, navigation routing can be implemented to include provision of a route alternative that is optimized in response to safety and/or driver stress profile factors. This can be implemented in addition to provision of route alternatives based on expected drive times, speed limits, traffic factors, etc., and the safety and/or stress based alternative can also consider these types of factors as well, since they may also contribute to lower safety and/or higher driver stress, particularly based on response to those factors on the safety and/or stress of the particular driver. Driver sensitivity profile development and updating such as, for example, implemented in optimization 300 of FIG. 3 and/or updating 400 of FIG. 4, can also be based on these and/or other factors.

In some embodiments, a navigation system develops a driving route based on minimum time, maximum safety, and/or minimum stress using optimization based on a driver safety profile and/or a driver stress profile. In some embodiments, route optimization is implemented in a manner similar to mapping software used to provide a route based on options such as least travel time, most time on highways, etc. However, in some embodiments, an additional route alternative is available for a navigation system in which a route alternative is available that provides a route based on safety and/or driver stress profile factors instead of or in addition to other routes such as, for example, those based on expected drive time, speed limits, traffic factors, etc. In some embodiments, driver safety and/or sensitivity profile development and updating is included in optimizing a route based on driver safety and/or stress profile factors. In some embodiments, a navigation system provides alternate routes for a driver to choose between. For example, alternate routes available to be chosen by a driver according to some embodiments can include routes based on minimum time, most time on highways, shortest distance, no time on tollways, etc. in addition to options optimized for safety and/or least driving stress.

In some embodiments, one or more of the following is considered in determining a route option based on driving stress:

Neighborhood crime rates (for example, in some embodiments, using one or more police and/or county databases) in order for a driver to avoid dangerous areas;

Intersections and/or streets with high accident occurrence rates (for example, in some embodiments, using one or more police and/or county databases);

Intersections with high rates of red light violations (for example, in some embodiments, using one or more police databases);

Streets with high rates of speeding drivers (for example, in some embodiments, using one or more police and/or traffic flow databases);

Street widths (for example, in some embodiments, using one or more county or other governmental record databases and/or other databases such as Google® street view);

Roundabouts or other unusual traffic patterns (for example, in some embodiments, using one or more map databases);

Blind turns and/or intersections (for example, in some embodiments, using one or more databases such as Google® street view and/or police accident databases).

In some embodiments, a navigation system generates recommendations in order to increase safety and/or reduce stress. This is implemented in some embodiments, for example, using predictive information such as predictive information from a driving condition predictor (for example, such as driving condition predictor 110 of FIG. 1). In some embodiments, such recommendations can include, for example, the navigation system suggesting that the driver consider leaving 30 minutes earlier or 30 minutes later in order to avoid traffic, waiting for a certain amount of time until weather conditions improve (such as fog clearing), etc.

In some embodiments, different routes can be graded by safety level and/or stress level on a quantitative or qualitative scale similar to a drive time estimate. In some embodiments, this grading is implemented using a road safety and/or stress level evaluator (for example, using road stress level evaluator 114 of FIG. 1).

In some embodiments, a navigation system can inform a driver whether or not a recommended higher safety and/or lower stress route is significantly better than another route (such as a fastest time route, for example). In some embodiments, this comparison can be qualitative. In some embodiments, this comparison can be quantitative.

In some embodiments, individual users can prioritize their particular needs. For example, in some embodiments, a driver sensitivity profile (such as, for example, driver sensitivity profile 116 of FIG. 1) can include direct user input of preferences. For example, a driver might input a preference to always avoid roads with bike lanes regardless of estimated safety and/or stress data from sensors, or always avoid roads with a certain level or higher of accident or traffic rates, etc.

In some embodiments, driver safety and/or stress scores are stored in a database and pulled from that database. In some embodiments, driver safety and/or stress scores are not necessarily pulled from a database. In some embodiments, safety and/or stress scores are computed by a navigation system (for example, computed locally and/or remotely) based on road conditions (for example, based on traffic conditions, road hazard conditions such as, for example, construction roadwork, icy roads, snowy roads, wet roads, etc., and/or databases such as static databases and/or crime rate databases). In some embodiments, one or more of a navigation system such as navigation system 100, navigation system 200, and/or driver profile optimization 300 and/or driver stress profile updating 400 are used to compute safety and/or stress scores. In some embodiments, stress scores are computed using navigation system 100 and/or navigation system 200 based on road conditions and/or using driver stress profile optimization 400.

In some embodiments, for example, if road A has one mile of a high crime rate street and five miles of a normal safe and/or no stress driving conditions road, while road B has one mile of icy conditions, two miles of driving congestion, and five miles of normal safety and/or no stress driving conditions road, one driver might have a different safety and/or stress routing than another driver. For example, driver 1 might have a driver safety and/or stress profile of a 90% safety/stress weight factor to crime rate, a 10% safety/stress weight factor to icy conditions, a 0% safety/stress weight factor to road congestion, and a 0% safety/stress weight factor to total trip distance, and driver 2 might have a driver safety/stress profile of a 10% safety/stress weight factor to crime rate, a 10% safety/stress weight factor to icy conditions, an 80% safety/stress weight factor to road congestion, and a 0% safety/stress weight factor to total trip distance. In this situation, safety and/or stress scores for driver 1 might indicate a lowest safety do/or highest stress on road A and safety and/or stress scores for driver 2 might indicate a lowest safety and/or highest stress on road B.

In some embodiments, an equation can be used to determine safety and/or stress scores for various drivers. For example, safety and/or stress scores may be calculated according to some embodiments as follows:

TS=T1*W1+T2*W2+T3*W3+ . . . +TX*WX (EQUATION 1)

Where TS is total stress and/or total safety, T1 is time spent in safety and/or stress level 1, W1 is weight factor of safety and/or stress level 1, T2 is time spent in safety and/or stress level 2, W2 is weight factor of safety and/or stress level 2, T3 is time spent in safety and/or stress level 3, W3 is weight factor of safety and/or stress level 3, . . . , TX is time spent in safety and/or stress level X, and WX is weight factor of safety and/or stress level X.

In some embodiments, various other ways can be used to determine safety and/or stress scores for various drivers. For example, safety and/or stress scores may be calculated using any way emphasizing safety level, stress level and/or time spent. For example, a non-linear calculation may be used to determine safety and/or stress scores according to some embodiments. For example, safety and/or stress scores may be calculated according to some embodiments as follows:

TS=T1*(W1)²+T2*(W2)²+T3*(W3)²+T2*(W2)²+ . . . +TX*(WX)² (EQUATION 2)

In some embodiments, safety and/or stress scores are calculated in other ways. In some embodiments, safety and/or stress score calculation can be adjusted and fine-tuned by comparing computed safety and/or stress to actual safety and/or stress measurements of the driver during each trip. In this manner, the accurate safety and/or stress score calculation can be improved over time.

In some embodiments, for example, if a person gave a factor a low safety weight and/or a high stress weight (for example, if the person gave high crime areas in general or a particular high crime area a low safety weight and/or a high stress weight), safety and/or stress navigation can provide that factor (for example, a particular area and/or a particular type of safety and/or stress) a low safety weight and/or a high stress weight for those factors. In some embodiments, if the safety and/or stress navigation routing system determined (and/or learned) that every time passing a particular type of road (for example, an icy road) that the safety level decreases and/or that the driver's stress increases (for example, by measuring that the driver's heartbeat increases, face gets nervous, etc.), safety and/or stress navigation can provide that factor a low safety weight and/or a high stress weight. In some embodiments, for example, safety weights and/or stress weights can be determined based on conditions such as road conditions, current user driving, past user driving (for example, using a user profile), etc.

In some embodiments, road conditions are pulled (for example, from a database in system 100 and/or system 200), a driver profile is pulled (for example, using system 100, system 200, and/or driver profile updating 400), and a score is computed (for example, using Equation 1, Equation 2, system 100, and/or system 200, etc.).

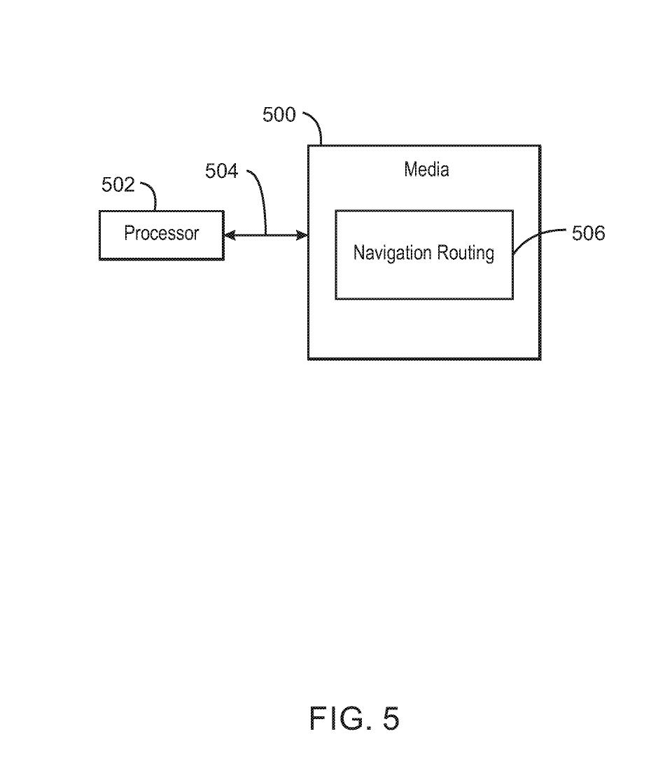

FIG. 5 is a block diagram of an example of a processor and one or more tangible, non-transitory computer readable media for stress based navigation. The one or more tangible, non-transitory, computer-readable media 500 may be accessed by the processor 502 over a computer interconnect 504. Furthermore, the one or more tangible, non-transitory, computer-readable media 500 may include code to direct the processor 502 to perform operations as described herein. In some embodiments, processor 502 is one or more processors. In some embodiments, processor 502 can perform similarly to (and/or the same as) processor 220 of FIG. 2, and can perform some or all of the same functions as can be performed by processor 220.

Various components discussed in this specification may be implemented using software components. These software components may be stored on the one or more tangible, non-transitory, computer-readable media 500, as indicated in FIG. 5. For example, navigation routing 506 (for example, navigation routing instructions) may be adapted to direct the processor 502 to perform one or more of any of the operations described in this specification and/or in reference to FIG. 1, FIG. 2, FIG. 3 or FIG. 4. For example, navigation routing 506 can be adapted to direct processor 502 to perform safety and/or stress based navigation routing, routing optimization, and/or safety and/or stress profile updating as described herein.

It is to be understood that any suitable number of the software components shown in FIG. 5 may be included within the one or more tangible, non-transitory computer-readable media 500. Furthermore, any number of additional software components not shown in FIG. 5 may be included within the one or more tangible, non-transitory, computer-readable media 500, depending on the specific application.

Some embodiments have been referred to herein as stress based navigation, and/or as relating to stress based routing, stress based factors, stress levels, etc. It is recognized that any embodiments referred to herein as being related to stress based navigation, routing, etc. can also be referred to as being related to safety based navigation, routing, etc. Some embodiments have been described herein as being related to the driver's safety and/or stress (and/or stress levels). However, some embodiments are related to anyone's safety (for example, driver safety, passenger safety, and/or safety of others that are neither passengers nor drivers, and/or are drivers and/or passengers of other vehicles). Further, some embodiments can relate to stress and/or stress levels of one or more passengers, either instead of or in addition to relating to stress and/or stress levels of the driver.

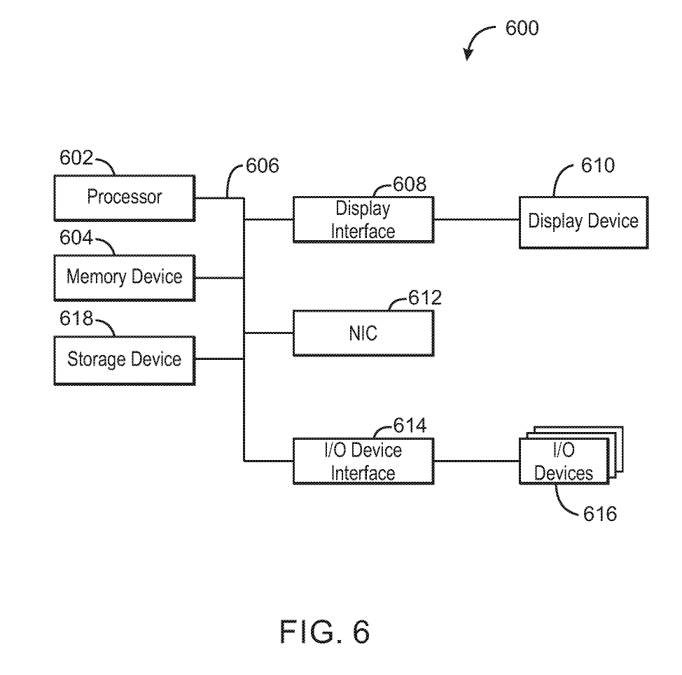

FIG. 6 is a block diagram of an example of a computing device 600 that can implement navigation routing (for example, stress based navigation routing). In some embodiments, computing device 600 is a navigation device (for example, a GPS device). In some embodiments, computing device can be included in a vehicle (for example, in an automobile). In some embodiments, any portion of the circuits and/or systems illustrated in any one or more of FIGS. 1-5, and any of the embodiments described herein can be included in and/or be implemented by computing device 600. The computing device 600 may be, for example, a navigation device, a GPS device, a mobile phone, mobile device, handset, laptop computer, desktop computer, or tablet computer, among others. The computing device 600 may include a processor 602 that is adapted to execute stored instructions, as well as a memory device 604 (and/or storage device 604) that stores instructions that are executable by the processor 602. The processor 602 can be a single core processor, a multi-core processor, a computing cluster, or any number of other configurations. For example, processor 602 can be an Intel® processor such as an Intel® Celeron, Pentium, Core, Core i3, Core i5, or Core i7 processor. In some embodiments, processor 602 can be an Intel® x86 based processor. In some embodiments, processor 602 can be an ARM based processor. The memory device 604 can be a memory device and/or a storage device, and can include volatile storage, non-volatile storage, random access memory, read only memory, flash memory, or any other suitable memory or storage systems. The instructions that are executed by the processor 602 may also be used to implement navigation routing as described in this specification.

The processor 602 may also be linked through the system interconnect 606 (e.g., PCI®, PCI-Express®, NuBus, etc.) to a display interface 608 adapted to connect the computing device 600 to a display device 610. The display device 610 may include a display screen that is a built-in component of the computing device 600. The display device 610 may also include a computer monitor, television, or projector, among others, that is externally connected to the computing device 600. The display device 610 can include light emitting diodes (LEDs), organic light emitting diodes (OLEDs), and/or micro-LEDs (μLEDs), among others.

In some embodiments, the display interface 608 can include any suitable graphics processing unit, transmitter, port, physical interconnect, and the like. In some examples, the display interface 608 can implement any suitable protocol for transmitting data to the display device 610. For example, the display interface 608 can transmit data using a high-definition multimedia interface (HDMI) protocol, a DisplayPort protocol, or some other wired or wireless protocol or communication link, and the like

In addition, one or more network interface controllers (also referred to herein as a NIC) 612 may be adapted to connect the computing device 600 through the system interconnect 606 to one or more networks or devices (not depicted). The network (not depicted) may be a cellular network, a radio network, a wide area network (WAN), a local area network (LAN), or the Internet, among others. In some embodiments, one or more NIC 612 can include a wireless device to connect to a GPS network, and/or to one or more satellites (for example, one or more GPS satellites).

The processor 602 may be connected through system interconnect 606 to an input/output (I/O) device interface 614 adapted to connect the computing host device 600 to one or more I/O devices 616. The I/O devices 616 may include, for example, a keyboard and/or a pointing device, where the pointing device may include a touchpad or a touchscreen, among others. The I/O devices 616 may be built-in components of the computing device 600, or may be devices that are externally connected to the computing device 600.

In some embodiments, the processor 602 may also be linked through the system interconnect 606 to a storage device 618 that can include a hard drive, a solid state drive (SSD), a magnetic drive, an optical drive, a USB flash drive, an array of drives, or any other type of storage, including combinations thereof. In some embodiments, the storage device 618 can include any suitable applications. In some embodiments, the storage device 618 can include a basic input/output system (BIOS).

It is to be understood that the block diagram of FIG. 6 is not intended to indicate that the computing device 600 is to include all of the components shown in FIG. 6. Rather, the computing device 600 can include fewer or additional components not illustrated in FIG. 6 (e.g., additional memory components, embedded controllers, additional modules, additional network interfaces, etc.) In some embodiments, databases and/or storage devices described herein (for example, databases 104, 106, history archive 112, driver sensitivity profile 116, etc.) can be coupled within computing device 600 (for example, as a storage device such as storage device 618, and/or can be connected to computing device 600, for example, using NIC 612). In some embodiments, sensors such as vehicle sensors 108) can be coupled to the computing device 600 (for example, as one or more I/O devices 616). In some embodiments, sensors such as, for example, one or more rain sensors, vehicle sensors, cameras, audio sensors, steering wheel sensors, etc. can be included in computing device 600 (for example, as one or more I/O devices 616).

Reference in the specification to “one embodiment” or “an embodiment” or “some embodiments” of the disclosed subject matter means that a particular feature, structure, or characteristic described in connection with the embodiment is included in at least one embodiment of the disclosed subject matter. Thus, the phrase “in one embodiment” or “in some embodiments” may appear in various places throughout the specification, but the phrase may not necessarily refer to the same embodiment or embodiments.

### Example 1

In some examples, a navigation routing system includes at least one processor to provide a navigation route based on one or more stress levels of a driver of a vehicle.

### Example 2

In some examples, in the system of EXAMPLE 1, the at least one processor is to determine (and/or learn) one or more conditions that provide stress to the driver. The at least one processor is to provide the navigation route in response to the determined conditions.

### Example 3

In some examples, in the system of EXAMPLE 2, the one or more conditions include at least one of locations, physical conditions of the driver, road conditions, weather conditions, conditions within the vehicle, movements of the vehicle, a calendar of the driver, traffic conditions, and/or crime conditions.

### Example 4

In some examples, in the system of EXAMPLE 2, at least one sensor is to monitor the driver. The at least one processor is to determine (and/or learn) one or more conditions that provide stress to the driver in response to the at least one sensor.

### Example 5

In some examples, in the system of EXAMPLE 2, at least one sensor is to monitor the vehicle. The at least one processor is to determine (and/or learn) one or more conditions that provide stress to the driver in response to the at least one sensor.

### Example 6

In some examples, in the system of EXAMPLE 1, the at least one processor is to obtain one or more conditions that provide stress to the driver. The at least one processor is to obtain a profile of the driver. The at least one processor is to compute one or more stress scores of possible navigation routes in response to the one or more conditions and in response to the profile of the driver. The at least one processor is to provide the navigation route in response to the one or more stress scores.

### Example 7

In some examples, in the system of EXAMPLE 1, the at least one processor is to obtain one or more conditions that provide stress to the driver. The at least one processor is to compute one or more stress scores of possible navigation routes in response to the one or more conditions. The at least one processor is to provide the navigation route in response to the one or more stress scores.

### Example 8

In some examples, a navigation routing system includes a route stress navigator to provide a navigation route based on one or more stress levels of a driver of a vehicle.

### Example 9

In some examples, in the system of EXAMPLE 8, a stress level evaluator is to determine (and/or learn) conditions that provide stress to the driver. The route stress navigator is to provide the navigation route in response to the determined (and/or learned) conditions.

### Example 10

In some examples, in the system of EXAMPLE 9, the one or more conditions include at least one of locations, physical conditions of the driver, road conditions, weather conditions, conditions within the vehicle, movements of the vehicle, a calendar of the driver, traffic conditions, and/or crime conditions.

### Example 11

In some examples, in the system of EXAMPLE 9, at least one sensor is to monitor the driver. The stress level evaluator is to determine (and/or learn) the one or more conditions that provide stress to the driver in response to the at least one sensor.

### Example 12

In some examples, in the system of EXAMPLE 9, at least one sensor is to monitor the vehicle. The stress level evaluator is to determine (and/or learn) the one or more conditions that provide stress to the driver in response to the at least one sensor.

### Example 13

In some examples, in the system of EXAMPLE 8, a stress level evaluator is to obtain one or more conditions that provide stress to the driver and to obtain a profile of the driver. The route stress navigator is to compute one or more stress scores of possible navigation routes in response to the one or more conditions and in response to the profile of the driver. The route stress navigator is to provide the navigation route in response to the one or more stress scores.

### Example 14

In some examples, in the system of EXAMPLE 8, a stress level evaluator is to obtain one or more conditions that provide stress to the driver. The route stress navigator is to compute one or more stress scores of possible navigation routes in response to the one or more conditions. The route stress navigator is to provide the navigation route in response to the one or more stress scores.

### Example 15

In some examples, a navigation routing method includes providing a navigation route based on one or more stress levels of a driver of a vehicle.

### Example 16

In some examples, in the method of EXAMPLE 15, the method includes determining (and/or learning) one or more conditions that provide stress to the driver, and providing the navigation route in response to the determined (and/or learned) conditions.

### Example 17

In some examples, in the method of EXAMPLE 16, the one or more conditions include at least one of locations, physical conditions of the driver, road conditions, weather conditions, conditions within the vehicle, movements of the vehicle, a calendar of the driver, traffic conditions, and/or crime conditions.

### Example 18

In some examples, in the method of EXAMPLE 15, the method includes obtaining one or more conditions that provide stress to the driver, obtaining a profile of the driver, computing one or more stress scores of possible navigation routes in response to the one or more conditions and in response to the profile of the driver, and providing the navigation route in response to the one or more stress scores.

### Example 19

In some examples, in the method of EXAMPLE 15, the method includes obtaining one or more conditions that provide stress to the driver, computing one or more stress scores of possible navigation routes in response to the one or more conditions, and providing the navigation route in response to the one or more stress scores.

### Example 20

In some examples, a tangible, non-transitory computer readable medium for transmitting data includes a plurality of instructions. In response to being executed on a processor, the instructions cause the processor to provide a navigation route based on one or more stress levels of a driver of a vehicle.

### Example 21

In some examples, in the computer readable medium of EXAMPLE 20, the plurality of instructions, in response to being executed on a processor, cause the processor to determine (and/or learn) one or more conditions that provide stress to the driver, and provide the navigation route in response to the determined (and/or learned) conditions.

### Example 22

In some examples, in the computer readable medium of EXAMPLE 21, the one or more conditions include at least one of locations, physical conditions of the driver, road conditions, weather conditions, conditions within the vehicle, movements of the vehicle, a calendar of the driver, traffic conditions, and/or crime conditions.

### Example 23

In some examples, in the computer readable medium of EXAMPLE 20, the plurality of instructions, in response to being executed on a processor, cause the processor to obtain one or more conditions that provide stress to the driver, obtain a profile of the driver, compute one or more stress scores of possible navigation routes in response to the one or more conditions and in response to the profile of the driver, and provide the navigation route in response to the one or more stress scores.

### Example 24

In some examples, in the computer readable medium of EXAMPLE 20, the plurality of instructions, in response to being executed on a processor, cause the processor to obtain one or more conditions that provide stress to the driver, compute one or more stress scores of possible navigation routes in response to the one or more conditions, and provide the navigation route in response to the one or more stress scores.

### Example 25

In some examples, a navigation routing system includes means for providing a navigation route based on one or more stress levels of a driver of a vehicle.

### Example 26

In some examples, in the system of EXAMPLE 25, the system includes means for determining (and/or learning) one or more conditions that provide stress to the driver, and means for providing the navigation route in response to the determined (and/or learned) conditions.

### Example 27

In some examples, in the system of EXAMPLE 26, the one or more conditions include at least one of locations, physical conditions of the driver, road conditions, conditions within the vehicle, movements of the vehicle, a calendar of the driver, traffic conditions, and/or crime conditions.

### Example 28

In some examples, in the system of EXAMPLE 25, 26, or 27, including means for monitoring the driver, and means for determining (and/or learning) the one or more conditions that provide stress to the driver in response to the means for monitoring.

### Example 29

In some examples, in the system of EXAMPLE 25, 26, or 27, including means for monitoring the vehicle, and means for determining (and/or learning) the one or more conditions that provide stress to the driver in response to the means for monitoring.

### Example 30

In some examples, in the system of EXAMPLE 25, 26, or 27, including means for obtaining one or more conditions that provide stress to the driver, means for obtaining a profile of the driver, means for computing one or more stress scores of possible navigation routes in response to the one or more conditions and in response to the profile of the driver, and means for providing the navigation route in response to the one or more stress scores.

### Example 31

In some examples, in the system of EXAMPLE 25, 26, or 27, including means for obtaining one or more conditions that provide stress to the driver, means for computing one or more stress scores of possible navigation routes in response to the one or more conditions, and means for providing the navigation route in response to the one or more stress scores.

### Example 32

In some examples, a navigation routing system, includes at least one processor to provide a navigation route based on one or more stress levels of a driver of a vehicle.

### Example 33

In some examples, in the system of EXAMPLE 32, the at least one processor to learn one or more conditions that provide stress to the driver, and to provide the navigation route in response to the determined (and/or learned) conditions.

### Example 34

In some examples, in the system of EXAMPLE 33, the one or more conditions include at least one of locations, physical conditions of the driver, road conditions, weather conditions, conditions within the vehicle, movements of the vehicle, a calendar of the driver, traffic conditions, and/or crime conditions.

### Example 35

In some examples, in the system of EXAMPLE 32, 33, or 34, the system includes at least one sensor. The at least one processor is to determine (and/or learn) the one or more conditions that provide stress to the driver in response to the at least one sensor.

### Example 36

In some examples, in the system of EXAMPLE 32, 33, or 34, the at least one processor is to obtain one or more conditions that provide stress to the driver, compute one or more stress scores of possible navigation routes in response to the one or more conditions, and provide the navigation route in response to the one or more stress scores.

### Example 37

In some examples, a navigation routing system includes a route stress navigator to provide a navigation route based on one or more stress levels of a driver of a vehicle.

### Example 38

In some examples, in the system of EXAMPLE 37, a stress level evaluator is to learn one or more conditions that provide stress to the driver. The route stress navigator is to provide the navigation route in response to the determined (and/or learned) conditions.

### Example 39

In some examples, in the system of EXAMPLE 37 or 38, the system includes at least one sensor. The stress level evaluator is to determine (and/or learn) the one or more conditions that provide stress to the driver in response to the at least one sensor.

### Example 40

In some examples, in the system of EXAMPLE 37 or 38, a stress level evaluator is to obtain one or more conditions that provide stress to the driver. The route stress navigator is to compute one or more stress scores of possible navigation routes in response to the one or more conditions. The route stress navigator is to provide the navigation route in response to the one or more stress scores.

### Example 41

In some examples, a navigation routing method includes providing a navigation route based on one or more stress levels of a driver of a vehicle.

### Example 42

In some examples, in the method of EXAMPLE 41, the method includes determining (and/or learning) one or more conditions that provide stress to the driver, and providing the navigation route in response to the determined (and/or learned) conditions.

### Example 43

In some examples, in the method of EXAMPLE 41 or 42, the method includes obtaining one or more conditions that provide stress to the driver, obtaining a profile of the driver, computing one or more stress scores of possible navigation routes in response to the one or more conditions and in response to the profile of the driver, and providing the navigation route in response to the one or more stress scores.

### Example 44

In some examples, in the method of EXAMPLE 41 or 42, the method including obtaining one or more conditions that provide stress to the driver, computing one or more stress scores of possible navigation routes in response to the one or more conditions, and providing the navigation route in response to the one or more stress scores.

### Example 45

In some examples, a tangible, non-transitory computer readable medium is for transmitting data. The medium includes a plurality of instructions that, in response to being executed on a processor, cause the processor to provide a navigation route based on one or more stress levels of a driver of a vehicle.

### Example 46

In some examples, in the computer readable medium of EXAMPLE 45, the plurality of instructions, in response to being executed on a processor, cause the processor to determine (and/or learn) one or more conditions that provide stress to the driver, and to provide the navigation route in response to the determined (and/or learned) conditions.

### Example 47

In some examples, in the computer readable medium of EXAMPLE 46, the one or more conditions include at least one of locations, physical conditions of the driver, road conditions, weather conditions, conditions within the vehicle, movements of the vehicle, a calendar of the driver, traffic conditions, and/or crime conditions.

### Example 48

In some examples, in the computer readable medium of EXAMPLE 45, 46, or 47, the plurality of instructions, in response to being executed on a processor, cause the processor to obtain one or more conditions that provide stress to the driver, to obtain a profile of the driver, to compute one or more stress scores of possible navigation routes in response to the one or more conditions and in response to the profile of the driver, and to provide the navigation route in response to the one or more stress scores.

### Example 49

In some examples, in the computer readable medium of EXAMPLE 45, the plurality of instructions, in response to being executed on a processor, cause the processor to obtain one or more conditions that provide stress to the driver, to compute one or more stress scores of possible navigation routes in response to the one or more conditions, and to provide the navigation route in response to the one or more stress scores.

### Example 50

In some examples, a navigation routing system includes at least one processor to provide a navigation route based on one or more stress levels of a driver of a vehicle.

### Example 51

In some examples, in the system of EXAMPLE 50, the at least one processor to determine (and/or learn) one or more conditions that provide stress to the driver, and to provide the navigation route in response to the determined (and/or learned) conditions.

### Example 52

In some examples, in the system of EXAMPLE 51, the one or more conditions include at least one of locations, physical conditions of the driver, road conditions, weather conditions, conditions within the vehicle, movements of the vehicle, a calendar of the driver, traffic conditions, and/or crime conditions.

### Example 53

In some examples, in the system of any of EXAMPLES 50-52, at least one sensor is to monitor the driver. The at least one processor is to determine (and/or learn) the one or more conditions that provide stress to the driver in response to the at least one sensor.

### Example 54

In some examples, in the system of any of EXAMPLES 50-52, at least one sensor is to monitor the vehicle. The at least one processor is to learn the one or more conditions that provide stress to the driver in response to the at least one sensor.

### Example 55

In some examples, in the system of any of EXAMPLES 50-52, the at least one processor is to obtain one or more conditions that provide stress to the driver, obtain a profile of the driver, compute one or more stress scores of possible navigation routes in response to the one or more conditions and in response to the profile of the driver, and provide the navigation route in response to the one or more stress scores.

### Example 56

In some examples, in the system of any of EXAMPLES 50-52, the at least one processor is to obtain one or more conditions that provide stress to the driver, compute one or more stress scores of possible navigation routes in response to the one or more conditions, and provide the navigation route in response to the one or more stress scores.

### Example 57

In some examples, a navigation routing system, includes a route stress navigator to provide a navigation route based on one or more stress levels of a driver of a vehicle.

### Example 58

In some examples, in the system of EXAMPLE 57, a stress level evaluator is to determine (and/or learn) one or more conditions that provide stress to the driver. The route stress navigator is to provide the navigation route in response to the determined (and/or learned) conditions.

### Example 59

In some examples, in the system of EXAMPLE 57, a stress level evaluator is to obtain one or more conditions that provide stress to the driver. The route stress navigator is to compute one or more stress scores of possible navigation routes in response to the one or more conditions. The route stress navigator is to provide the navigation route in response to the one or more stress scores.

### Example 60

In some examples, in the system of EXAMPLE 57, a stress level evaluator is to obtain one or more conditions that provide stress to the driver and to obtain a profile of the driver. The route stress navigator is to compute one or more stress scores of possible navigation routes in response to the one or more conditions and in response to the profile of the driver, and to provide the navigation route in response to the one or more stress scores.

### Example 61

In some examples, in the system of any of EXAMPLES 58-60, the one or more conditions include at least one of locations, physical conditions of the driver, road conditions, weather conditions, conditions within the vehicle, movements of the vehicle, a calendar of the driver, traffic conditions, and/or crime conditions.

### Example 62

In some examples, in the system of any of EXAMPLES 57-60, at least one sensor is to monitor the driver. The stress level evaluator is to determine (and/or learn) the one or more conditions that provide stress to the driver in response to the at least one sensor.

### Example 63

In some examples, in the system of any of EXAMPLES 57-60, at least one sensor is to monitor the vehicle. The stress level evaluator is to determine (and/or learn) the one or more conditions that provide stress to the driver in response to the at least one sensor.

### Example 64

In some examples, a navigation routing method includes providing a navigation route based on one or more stress levels of a driver of a vehicle.

### Example 65

In some examples, in the method of EXAMPLE 64, the method includes determining (and/or learning) one or more conditions that provide stress to the driver, and providing the navigation route in response to the determined (and/or learned) conditions.

### Example 66

In some examples, in the method of EXAMPLE 64, the method includes obtaining one or more conditions that provide stress to the driver, obtaining a profile of the driver, computing one or more stress scores of possible navigation routes in response to the one or more conditions and in response to the profile of the driver, and providing the navigation route in response to the one or more stress scores.

### Example 67

In some examples, in the method of EXAMPLE 64, the method includes obtaining one or more conditions that provide stress to the driver, computing one or more stress scores of possible navigation routes in response to the conditions, and providing the navigation route in response to the one or more stress scores.

### Example 68

In some examples, in the method of any of EXAMPLES 65-67, the one or more conditions include at least one of locations, physical conditions of the driver, road conditions, weather conditions, conditions within the vehicle, movements of the vehicle, a calendar of the driver, traffic conditions, and/or crime conditions.

### Example 69

In some examples, in the method of any of EXAMPLES 64-67, the method includes monitoring the driver, and determining (and/or learning) the one or more conditions that provide stress to the driver in response to the monitoring.

### Example 70

In some examples, in the method of any of EXAMPLES 64-67, the method includes monitoring the vehicle, and determining (and/or learning) the one or more conditions that provide stress to the driver in response to the monitoring.

### Example 71

In some examples, in the method of EXAMPLE 69, the method includes monitoring the vehicle, and determining (and/or learning) the one or more conditions that provide stress to the driver in response to the monitoring.

### Example 72

In some examples, a machine-readable medium includes code, when executed, to cause a machine to perform the method of any one of EXAMPLES 64-67.

### Example 73

In some examples, a navigation routing system includes means for providing a navigation route based on one or more stress levels of a driver of a vehicle.

### Example 74

In some examples, in the system of EXAMPLE 73, the system includes means for determining (and/or learning) one or more conditions that provide stress to the driver, and means for providing the navigation route in response to the determined (and/or learned) conditions.

### Example 75

In some examples, a navigation routing method includes providing a navigation route based on one or more stress levels of a driver of a vehicle.

### Example 76

In some examples, in the method of EXAMPLE

75, the method includes determining (and/or learning) one or more conditions that provide stress to the driver, and/or providing the navigation route in response to the determined (and/or learned) conditions.

### Example 77

In some examples, in the method of any of EXAMPLES 75-76, the method includes obtaining one or more conditions that provide stress to the driver, and/or obtaining a profile of the driver, and/or computing one or more stress scores of possible navigation routes in response to the one or more conditions and in response to the profile of the driver, and/or providing the navigation route in response to the one or more stress scores.

### Example 78

In some examples, in the method of any of EXAMPLES 75-77, the method including obtaining one or more conditions that provide stress to the driver, and/or computing one or more stress scores of possible navigation routes in response to the one or more conditions, and/or providing the navigation route in response to the one or more stress scores.

### Example 79

In some examples, in the method of any of EXAMPLES 76-78, the one or more conditions include at least one of locations, and/or physical conditions of the driver, and/or road conditions, and/or weather conditions, and/or conditions within the vehicle, and/or movements of the vehicle, and/or a calendar of the driver, and/or traffic conditions, and/or crime conditions.

### Example 80

In some examples, in the method of any of EXAMPLES 75-79, the method including monitoring the driver, and/or determining (and/or learning) the one or more conditions that provide stress to the driver in response to the monitoring.

### Example 81

In some examples, in the method of any of EXAMPLES 75-80, the method including monitoring the vehicle, and/or determining (and/or learning) the one or more conditions that provide stress to the driver in response to the monitoring.

### Example 82

In some examples, an apparatus including means to perform a method as in any of EXAMPLES 75-81.

### Example 83

In some examples, in the apparatus of EXAMPLE 82, at least one processor to perform the method.

### Example 84

In some examples, a machine-readable storage includes machine-readable instructions, when executed, to implement a method or realize an apparatus as in any of EXAMPLES 75-83.

Although an example embodiment of the disclosed subject matter is described with reference to the drawings, persons of ordinary skill in the art will readily appreciate that many other ways of implementing the disclosed subject matter may alternatively be used. For example, the order of execution of the blocks in flow diagrams may be changed, and/or some of the blocks in block/flow diagrams described may be changed, eliminated, or combined. Additionally, some of the circuit and/or block elements may be changed, eliminated, or combined.

In the preceding description, various aspects of the disclosed subject matter have been described. For purposes of explanation, specific numbers, systems and configurations were set forth in order to provide a thorough understanding of the subject matter. However, it is apparent to one skilled in the art having the benefit of this disclosure that the subject matter may be practiced without the specific details. In other instances, well-known features, components, or modules were omitted, simplified, combined, or split in order not to obscure the disclosed subject matter.

Various embodiments of the disclosed subject matter may be implemented in hardware, firmware, software, or combination thereof, and may be described by reference to or in conjunction with program code, such as instructions, functions, procedures, data structures, logic, application programs, design representations or formats for simulation, emulation, and fabrication of a design, which when accessed by a machine results in the machine performing tasks, defining abstract data types or low-level hardware contexts, or producing a result.

Program code may represent hardware using a hardware description language or another functional description language which essentially provides a model of how designed hardware is expected to perform. Program code may be assembly or machine language or hardware-definition languages, or data that may be compiled and/or interpreted. Furthermore, it is common in the art to speak of software, in one form or another as taking an action or causing a result. Such expressions are merely a shorthand way of stating execution of program code by a processing system which causes a processor to perform an action or produce a result.

Program code may be stored in, for example, volatile and/or non-volatile memory, such as storage devices and/or an associated machine readable or machine accessible medium including solid-state memory, hard-drives, floppy-disks, optical storage, tapes, flash memory, memory sticks, digital video disks, digital versatile discs (DVDs), etc., as well as more exotic mediums such as machine-accessible biological state preserving storage. A machine readable medium may include any tangible mechanism for storing, transmitting, or receiving information in a form readable by a machine, such as antennas, optical fibers, communication interfaces, etc. Program code may be transmitted in the form of packets, serial data, parallel data, etc., and may be used in a compressed or encrypted format.

Program code may be implemented in programs executing on programmable machines such as mobile or stationary computers, personal digital assistants, set top boxes, cellular telephones and pagers, and other electronic devices, each including a processor, volatile and/or non-volatile memory readable by the processor, at least one input device and/or one or more output devices. Program code may be applied to the data entered using the input device to perform the described embodiments and to generate output information. The output information may be applied to one or more output devices. One of ordinary skill in the art may appreciate that embodiments of the disclosed subject matter can be practiced with various computer system configurations, including multiprocessor or multiple-core processor systems, minicomputers, mainframe computers, as well as pervasive or miniature computers or processors that may be embedded into virtually any device. Embodiments of the disclosed subject matter can also be practiced in distributed computing environments where tasks may be performed by remote processing devices that are linked through a communications network.

Although operations may be described as a sequential process, some of the operations may in fact be performed in parallel, concurrently, and/or in a distributed environment, and with program code stored locally and/or remotely for access by single or multi-processor machines. In addition, in some embodiments the order of operations may be rearranged without departing from the spirit of the disclosed subject matter. Program code may be used by or in conjunction with embedded controllers.

While the disclosed subject matter has been described with reference to illustrative embodiments, this description is not intended to be construed in a limiting sense. Various modifications of the illustrative embodiments, as well as other embodiments of the subject matter, which are apparent to persons skilled in the art to which the disclosed subject matter pertains are deemed to lie within the scope of the disclosed subject matter. For example, in each illustrated embodiment and each described embodiment, it is to be understood that the diagrams of the figures and the description herein is not intended to indicate that the illustrated or described devices include all of the components shown in a particular figure or described in reference to a particular figure. In addition, each element may be implemented with logic, wherein the logic, as referred to herein, can include any suitable hardware (e.g., a processor, among others), software (e.g., an application, among others), firmware, or any suitable combination of hardware, software, and firmware, for example.

What is claimed is:

1. A navigation routing system, comprising:
storage to store instructions executable by at least one processor;
at least one processor to execute the instructions to provide a navigation route based on one or more stress levels of a driver of a vehicle.

2. The system of claim 1, the at least one processor to determine one or more conditions that provide stress to the driver, and to provide the navigation route in response to the determined conditions.

3. The system of claim 2, wherein the one or more conditions include at least one of locations, physical conditions of the driver, road conditions, weather conditions, conditions within the vehicle, movements of the vehicle, a calendar of the driver, traffic conditions, and/or crime conditions.

4. The system of claim 2, comprising at least one sensor to monitor the driver, the at least one processor to determine one or more conditions that provide stress to the driver in response to the at least one sensor.

5. The system of claim 2, comprising at least one sensor to monitor the vehicle, the at least one processor to determine one or more conditions that provide stress to the driver in response to the at least one sensor.

6. The system of claim 1, the at least one processor to:
obtain one or more conditions that provide stress to the driver;
obtain a profile of the driver;
compute one or more stress scores of possible navigation routes in response to the one or more conditions and in response to the profile of the driver; and
provide the navigation route in response to the one or more stress scores.

7. The system of claim 1, the at least one processor to:
obtain one or more conditions that provide stress to the driver;
compute one or more stress scores of possible navigation routes in response to the one or more conditions; and
provide the navigation route in response to the one or more stress scores.

8. A navigation routing system, comprising:
a stress level evaluator to determine one or more stress levels of a driver of a vehicle; and
a route stress navigator to provide a navigation route based on the one or more stress levels of the driver of the vehicle.

9. The system of claim 8, the stress level evaluator to determine one or more conditions that provide stress to the driver, the route stress navigator to provide the navigation route in response to the determined conditions.

10. The system of claim 9, wherein the one or more conditions include at least one of locations, physical conditions of the driver, road conditions, weather conditions, conditions within the vehicle, movements of the vehicle, a calendar of the driver, traffic conditions, and/or crime conditions.

11. The system of claim 9, comprising at least one sensor to monitor the driver, the stress level evaluator to determine one or more conditions that provide stress to the driver in response to the at least one sensor.

12. The system of claim 9, comprising at least one sensor to monitor the vehicle, the stress level evaluator to determine one or more conditions that provide stress to the driver in response to the at least one sensor.

13. The system of claim 8, the stress level evaluator to obtain one or more conditions that provide stress to the driver and to obtain a profile of the driver, the route stress navigator to compute one or more stress scores of possible navigation routes in response to the one or more conditions and in response to the profile of the driver, and to provide the navigation route in response to the one or more stress scores.

14. The system of claim 8, the stress level evaluator to obtain one or more conditions that provide stress to the driver, the route stress navigator to compute one or more stress scores of possible navigation routes in response to the one or more conditions, and to provide the navigation route in response to the one or more stress scores.

15. A navigation routing method, comprising:
providing a navigation route based on one or more stress levels of a driver of a vehicle.

16. The method of claim 15, comprising determining one or more conditions that provide stress to the driver, and providing the navigation route in response to the determined conditions.

17. The method of claim 16, wherein the one or more conditions include at least one of locations, physical conditions of the driver, road conditions, weather conditions, conditions within the vehicle, movements of the vehicle, a calendar of the driver, traffic conditions, and/or crime conditions.

18. The method of claim 15, comprising:
obtaining one or more conditions that provide stress to the driver;
obtaining a profile of the driver;
computing one or more stress scores of possible navigation routes in response to the one or more conditions and in response to the profile of the driver; and
providing the navigation route in response to the one or more stress scores.

19. The method of claim 15, comprising:
obtaining one or more conditions that provide stress to the driver;
computing one or more stress scores of possible navigation routes in response to the one or more conditions; and
providing the navigation route in response to the one or more stress scores.

20. One or more tangible, non-transitory computer readable media for transmitting data comprising a plurality of instructions that, in response to being executed on a processor, cause the processor to:
provide a navigation route based on one or more stress levels of a driver of a vehicle.

21. The one or more computer readable media of claim 20, the plurality of instructions, in response to being executed on a processor, cause the processor to:
determine one or more conditions that provide stress to the driver; and
provide the navigation route in response to the determined conditions.

22. The one or more computer readable media of claim 21, wherein the one or more conditions include at least one of locations, physical conditions of the driver, road conditions, weather conditions, conditions within the vehicle, movements of the vehicle, a calendar of the driver, traffic conditions, and/or crime conditions.

23. The one or more computer readable media of claim 20, the plurality of instructions, in response to being executed on a processor, cause the processor to:
obtain one or more conditions that provide stress to the driver;
obtain a profile of the driver;
compute one or more stress scores of possible navigation routes in response to the one or more conditions and in response to the profile of the driver; and
provide the navigation route in response to the one or more stress scores.

24. The one or more computer readable media of claim 20, the plurality of instructions, in response to being executed on a processor, cause the processor to:
obtain one or more conditions that provide stress to the driver;
compute one or more stress scores of possible navigation routes in response to the one or more conditions; and
provide the navigation route in response to the one or more stress scores.
